(19) United States
(12) Patent Application Publication
(10) Pub. No.: US 2024/0376324 A1
(43) Pub. Date: Nov. 14, 2024

(54) ANTIBACTERIAL COATING AND USE THEREOF

(71) Applicant: Anhui Medical University, Hefei City (CN)

(72) Inventors: Xiangyang LI, Hefei City (CN); Weirui NIU, Hefei City (CN); Jialong CHEN, Hefei City (CN); Hua QIU, Hefei City (CN); Yankai WANG, Hefei City (CN); Yuan LI, Hefei City (CN)

(73) Assignee: Anhui Medical University, Hefei City (CN)

(21) Appl. No.: 18/658,767

(22) Filed: May 8, 2024

(30) Foreign Application Priority Data
May 8, 2023 (CN) 202310511726.0

(51) Int. Cl. C09D 5/14; A01N 55/02; A01P 1/00; C09D 185/00

(52) U.S. Cl. CPC C09D 5/14; A01N 55/02; A01P 1/00; C09D 185/00

(57) ABSTRACT

Provided are an antibacterial coating and a preparation method and use thereof. The preparation method includes: dissolving catecholamine, bacitracin, and a metal ion in a tris(hydroxymethyl)aminomethane solution (Tris solution) to obtain a dissolved mixture, subjecting the dissolved mixture to reaction on a titanium sheet for 24 h at ambient temperature under an aerobic environment, then subjecting a surface of the titanium sheet to ultrasonic cleaning to remove a precipitate thereon, and conducting blow-drying to obtain the antibacterial coating.

### CROSS REFERENCE TO RELATED APPLICATION

This application claims the benefit and priority of Chinese Patent Application No. 202310511726.0 filed with the China National Intellectual Property Administration on May 8, 2023, the disclosure of which is incorporated by reference herein in its entirety as part of the present application.

### TECHNICAL FIELD

The present disclosure relates to the technical field of surface modification of dental implants, and in particular relates to an antibacterial coating and a preparation method thereof, and use of the same in surface modification of a dental implant.

### BACKGROUND

At present, dental implant has become one of the effective ways to repair dentition defects in oral clinics and has been accepted and recognized by increasing patients. However, implant-related infection remains one of the most serious complications after implant surgery. In the worst case, the implants generally become loose and fall off, and some of which require secondary surgery, causing psychological trauma and financial burden to the patients. Therefore, it is crucial to clarify the pathogenesis of peri-implant infection and explore methods to prevent peri-implant infection to reduce and prevent the occurrence of peri-implant infection and thereby improve the success rate of implantation and the service life of implants. Peri-implant infection is essentially an inflammatory damage that destroys the tissue around the implant, and can be divided into early infection and late infection based on the occurrence time. The early infection occurs before the implant is integrated with the bone (that is, during an initial healing period after the implant is implanted), and is mainly caused by bacterial invasion and surgical trauma, which may damage or hinder the integration of the implant with the bone. The late infection occurs after the implant is integrated with the bone and is mainly related to peri-implantitis. If the late infection is not treated in time, it would cause progressive damages to the formed implant-bone interface. Compared with the late infection, early postoperative infection of implants is the main cause of treatment failure. Since the oral environment is a multi-bacterial environment, it is extremely difficult to control the local bacterial load during surgery. In severe cases, the early postoperative infection of implants would lead to osteolysis around the implant, thus in turn causing the implant to loosen or even fall off. Accordingly, antibacterial treatment is one of the important factors to maintain the long-term stability of implants.

The microbiota in oral cavity may have a substantial influence on biofilm formation on newly-placed implants. In a comparative study of early-stage adherent bacteria around implants and teeth, Mirjam M. et al. found that Staphylococcus aureus accounts for a higher proportion of early-stage colonization of bacteria on the implant surface. Currently, there is increasing evidence that Staphylococcus aureus may be an important pathogen in some cases of implant periodontitis, and many studies have also reported the association of Staphylococcus aureus, Enterobacter, and Candida albicans with peri-implant inflammation.

Titanium and its alloys are considered the best implant materials due to their desirable biocompatibility, mechanical properties, and corrosion resistance. However, titanium-based materials are not antimicrobial. In the early stages of implantation, peri-implant infection and poor osseointegration cause loss of peri-implant supporting tissue, which is generally the main reason for treatment failure. Compared with natural teeth, implant dentures have their own special features. The junctional epithelium at the implant cuff (an oral soft tissue surrounding the gingiva of the implant) mainly acts as a barrier and forms a desirable biological seal around the implant. However, compared with natural teeth, there is a relatively thin epithelial attachment of junctional epithelium at the implant cuff, resulting in a relatively weak barrier effect. In addition, there are fewer blood vessels in the connective tissue around implants than in natural teeth, and a binding interface between implants and bone lacks periodontal ligament. When bacteria invade, there are low inflammatory responses in the binding interface, such that the immune defense capability may be correspondingly weak, allowing bacteria to accumulate and multiply in this interface, leading to peri-implant infections.

Antibiotic antibacterial coatings mainly rely on the unique antibacterial methods of various antibiotics to achieve antibacterial effects, and exhibit advantages such as high biocompatibility and targeted antibacterial properties, and the biggest disadvantage of development of drug resistance. Bacitracin is a widely used metallopeptide antibiotic produced by Bacillus subtilis and Bacillus licheniformis with strong bactericidal activity, mainly against Gram-positive bacteria. This antibiotic requires a divalent metal ion (such as Zn) to obtain its biological activity. Despite decades of widespread use, bacterial resistance to bacitracin remains rare. A recently published article has showed that resistance to antibiotic peptides occurs less frequently than resistance to conventional antibiotics. As a result, the bacitracin could serve as a prototype to better understand the structure and function relationships of antibiotic peptides and serve as a leading drug for the rational design of antibiotic peptides in the future. In addition, the success of chemical synthesis of bacitracin has also greatly reduced its use cost. In recent years, several metallopeptide families have been designed as model systems for exploring the structure and function of metalloproteins. Understanding the structural and functional relationships of the antibiotic peptide metallobacitracin also points to new directions for the rational design of metallopeptides as potential models for metalloproteins. Bacitracin could block cell wall synthesis by specifically binding to the synthetic precursor of peptidoglycan, the main substance of the cell wall. However, there is no obvious effect on eukaryotic animal cells, and thus the bacitracin could be bactericidal without cytotoxicity. Moreover, since being proposed in the 1980s, the bacitracin has not been found to produce drug resistance and thus is an ideal antibacterial substance.

Bacitracin molecule includes one cyclic dodecapeptide, one special thiazoline ring, one cyclic heptapeptide structure, and four D-amino acids. These unusual structural features protect the bacitracin from protease degradation. A “tail” of the bacitracin bends toward the seven-membered ring, bringing the thiazoline ring, Glu, and His into close proximity to form a potential metal-binding site in solution. Through an amino group on the bacitracin, a self-polymerizing substance norepinephrine (NE) that could be covalently combined with the bacitracin is found, and then through the coordination with Zn, positive and negative point attraction, the bacitracin is efficiently loaded on a coating surface by a one-step blending method.

In the present disclosure, a coating construction method suitable for loading and exerting the antibacterial function of bacitracin is found by analyzing the characteristics of bacitracin molecules. Through the charge interaction and covalent interaction between NE and bacitracin, as well as the coordination interaction between NE and Zn ions, a stable coating is formed through a one-step blending method, which not only achieves antimicrobial properties but also takes into account cytocompatibility, providing a new strategy for surface modification of implants with similar structures.

### SUMMARY

A technical problem to be solved by the present disclosure is to provide a coating that is suitable for loading bacitracin and exerting an antibacterial function of the bacitracin.

To achieve the above objects, the present disclosure provides an antibacterial coating, which is formed by polymerization of catecholamine, bacitracin, and a metal ion.

In some embodiments of the present disclosure, the present disclosure provides a method for preparing an antibacterial coating including: dissolving catecholamine, bacitracin, and a metal ion in a tris(hydroxymethyl)aminomethane solution (Tris solution) to obtain a dissolved mixture, subjecting the dissolved mixture to reaction on a titanium sheet for 24 h at ambient temperature under an aerobic environment, then subjecting a surface of the titanium sheet to ultrasonic cleaning to remove a precipitate thereon, and conducting blow-drying to obtain the antibacterial coating.

In some embodiments, the Tris solution has a pH value of 7.5 to 9.5.

In some embodiments, the aerobic environment is achieved by exposure to air.

In some embodiments, the catecholamine is norepinephrine (NE).

In some embodiments, the metal ion is provided by ZnCl₂.

In some embodiments, in the dissolved mixture, the NE has a concentration of 1 mg/mL to 5 mg/mL, the bacitracin has a concentration of 0.12 mg/mL to 1 mg/mL, and the ZnCl₂ has a concentration of 0.5 mg/mL to 2 mg/mL.

In further embodiments, in the dissolved mixture, the NE has a concentration of 3 mg/mL, the bacitracin has a concentration of 0.25 mg/mL, and the ZnCl₂ has a concentration of 1 mg/mL.

In some embodiments, the present disclosure provides use of the antibacterial coating as described in the above solutions in surface modification of an interventional medical device.

In some embodiments, the interventional medical device is a dental implant.

The antibacterial coating constructed according to the present disclosure shows desirable antibacterial properties against Staphylococcus aureus, with a bacteriostatic rate of 72%. Furthermore, the antibacterial coating is non-toxic to the human body and exhibits a desirable biocompatibility.

### BRIEF DESCRIPTION OF THE DRAWINGS

FIG. 1 shows test results of the anti-Staphylococcus aureus performance of antibacterial coatings prepared by one-step blending method and multi-step grafting method, respectively; where Group A represents the NE antibacterial coating (NE+bacitracin+Zn) prepared by multi-step grafting method; Group B represents dopamine (Dopa) antibacterial coating (Dopa+bacitracin+Zn) prepared by multi-step grafting method; Group C represents NE antibacterial coating (NE@bacitracin@Zn) prepared by one-step blending method; Group D represents a dopamine (Dopa) antibacterial coating (Dopa@bacitracin@Zn) prepared by one-step blending method; and Control group represents the blank control group;

FIG. 2 shows test results of the anti-Staphylococcus aureus performance of the antibacterial coatings prepared by various methods;

FIG. 3 shows test results of the anti-Staphylococcus aureus performance of NE@bacitracin@Zn antibacterial coatings prepared with different concentrations of bacitracin:

FIG. 4A shows test results of cck-8 proliferation of NE@bacitracin@Zn antibacterial coatings prepared with different concentrations of bacitracin;

FIG. 4B shows fluorescence staining results of MC3T3-E1 proliferation of NE@bacitracin@Zn antibacterial coatings prepared with different concentrations of bacitracin;

FIG. 5A shows test results of cck-8 proliferation of NE@bacitracin@Zn antibacterial coatings prepared with different concentrations of ZnCl₂; and

FIG. 5B shows fluorescence staining results of MC3T3-E1 proliferation of NE@bacitracin@Zn antibacterial coatings prepared with different concentrations of ZnCl₂.

### DETAILED DESCRIPTION OF THE EMBODIMENTS

Various examples of the present disclosure will be introduced below with reference to the accompanying drawings, such that the technical solutions could be understood clearly and easily. The present disclosure could be embodied by examples in many different forms, and the scope of the present disclosure is not limited to these examples mentioned herein.

### Example 1

In the present disclosure, a base material was selected first. NE was selected as the base material among many catecholamine materials, and was able to prepare a coating with antibacterial properties. Dopamine, a commonly used material for catecholamines, was used as a comparison material.

### 1. Preparation of Coatings:

1) One-step blending method: catecholamine, bacitracin, and ZnCl₂ were dissolved in a Tris solution (pH=8.5, 1.21 mg/mL) under alkaline conditions to obtain a dissolved mixture of the catecholamine (3 mg/mL), the bacitracin (0.25 mg/mL), and the ZnCl₂ (1 mg/mL), and the dissolved mixture were subjected to reaction on a titanium sheet for 24 h at ambient temperature (25° C.) in aerobic environment, and a surface of the titanium sheet was then subjected to ultrasonic cleaning to remove a precipitate thereon, and then subjected to blow-drying to obtain an antibacterial coating.
2) Multi-step grafting method: catecholamine was dissolved in a Tris solution (pH=8.5, 1.21 mg/mL) under alkaline conditions, and dissolved catecholamine (3 mg/mL) was subjected to reaction on a titanium sheet for 24 h at ambient temperature (25° C.) in aerobic environment, and a resulting system was then subjected to ultrasonic cleaning and blow-drying. Further, a dissolved bacitracin (0.25 mg/mL) in a Tris solution was subjected to reaction on the titanium sheet for 24 h under the same pH, temperature, and oxygen conditions, and a resulting system was then subjected to ultrasonic cleaning and blow-drying. Finally, a dissolved ZnCl₂ (1 mg/mL) in a Tris solution was subjected to reaction on the titanium sheet for 24 h under the same pH, temperature, and oxygen conditions, and a resulting system was then subjected to ultrasonic cleaning and blow-drying to obtain an antibacterial coating.

### 2. Evaluation of In Vitro Antibacterial Properties

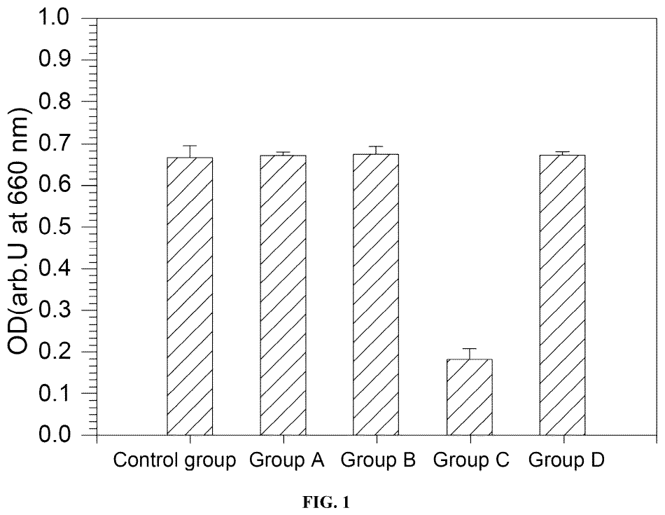

The anti-Staphylococcus aureus performance of the prepared catecholamine coatings was evaluated by turbidity method. Group A represents the NE antibacterial coating (NE+bacitracin+Zn) prepared by multi-step grafting method; Group B represents dopamine (Dopa) antibacterial coating (Dopa+bacitracin+Zn) prepared by multi-step grafting method; Group C represents NE antibacterial coating (NE@Obacitracin@Zn) prepared by one-step blending method; Group D represents a dopamine (Dopa) antibacterial coating (Dopa@bacitracin@Zn) prepared by one-step blending method; and Control group represents the blank control group. As shown in FIG. 1, only Group C coating exhibits a significant antibacterial property against Staphylococcus aureus.

### Example 2

In order to explore the relationship between coating loading mechanism and antibacterial performance, antibacterial coatings prepared by using various methods were prepared in the present disclosure.

### 1. Preparation of Coatings:

1) Blank control was prepared.
2) NE was dissolved in a Tris solution (pH=8.5, 1.21 mg/mL) under alkaline conditions, and dissolved NE (3 mg/mL) was subjected to reaction on a titanium sheet for 24 h at ambient temperature (25° C.) in aerobic environment, and then a resulting system was subjected to ultrasonic cleaning and blow-drying to obtain an NE coating.
3) NE and ZnCl₂ were dissolved in a Tris solution (pH=8.5, 1.21 mg/mL) under alkaline conditions, the dissolved mixture of NE (3 mg/mL) and ZnCl₂ (1 mg/mL) was subjected to reaction on a titanium sheet for 24 h at ambient temperature (25° C.) in aerobic environment, and then a resulting system was subjected to ultrasonic cleaning to obtain an NE@Zn coating.
4) Multi-step grafting method: NE and ZnCl₂ were dissolved in a Tris solution (pH=8.5, 1.21 mg/mL) under alkaline conditions, a dissolved mixture of NE (3 mg/mL) and ZnCl₂ (1 mg/mL) were subjected to reaction on a titanium sheet for 24 h at ambient temperature (25° C.) in aerobic environment, and then a resulting system was subjected to ultrasonic cleaning and blow-drying. Further, a dissolved bacitracin (0.25 mg/mL) in a Tris solution was subjected to reaction on the titanium sheet for 24 h under the same pH, temperature, and oxygen conditions, and then a resulting system was subjected to ultrasonic cleaning and blow-drying to obtain an NE@Zn+bacitracin antibacterial coating.
5) NE was dissolved in a Tris solution (pH=8.5, 1.21 mg/mL) under alkaline conditions, and a dissolved NE (3 mg/mL) was subjected to reaction on a titanium sheet for 24 h at ambient temperature (25° C.) in aerobic environment, and then a resulting system was subjected to ultrasonic cleaning and blow-drying. Further, a dissolved bacitracin (0.25 mg/mL) in a Tris solution was subjected to reaction on the titanium sheet for 24 h under the same pH, temperature, and oxygen conditions, and then a resulting system was subjected to ultrasonic cleaning and blow-drying to obtain an NE+bacitracin antibacterial coating.
6) NE and bacitracin were dissolved in a Tris solution (pH=8.5, 1.21 mg/mL) under alkaline conditions, and a dissolved mixture of NE (3 mg/mL) and bacitracin (0.25 mg/mL) were subjected to reaction on a titanium sheet for 24 h at ambient temperature (25° C.) in aerobic environment, and then a resulting system was subjected to ultrasonic cleaning and blow-drying to obtain an NE@bacitracin antibacterial coating.
7) Multi-step grafting method: NE and bacitracin were dissolved in a Tris solution (pH=8.5, 1.21 mg/mL) under alkaline conditions, and a dissolved mixture of NE (3 mg/mL) and bacitracin (0.25 mg/mL) were subjected to reaction on a titanium sheet for 24 h at ambient temperature (25° C.) in aerobic environment, and then a resulting system was subjected to ultrasonic cleaning and blow-drying. Further, a dissolved ZnCl₂ (1 mg/mL) in a Tris solution was subjected to reaction on the titanium sheet for 24 h under the same pH, temperature, and oxygen conditions, and then a resulting system was subjected to ultrasonic cleaning and blow-drying to obtain an NE@bacitracin+Zn antibacterial coating.
8) One-step blending method: an NE@bacitracin@Zn antibacterial coating was prepared according to Example 1.
9) NE was dissolved in a Tris solution (pH=8.5, 1.21 mg/mL) under alkaline conditions, and a dissolved NE (3 mg/mL) was subjected to reaction on a titanium sheet for 24 h at ambient temperature (25° C.) in aerobic environment, and then a resulting system was subjected to ultrasonic cleaning and blow-drying. Further, a dissolved ZnCl₂ in Tris solution (1 mg/mL) was subjected to reaction on the titanium sheet for 24 h under the same pH, temperature, and oxygen conditions, and then a resulting system was subjected to ultrasonic cleaning and blow-drying to obtain an NE+bacitracin antibacterial coating.
10) Multi-step grafting method: an NE+bacitracin+Zn antibacterial coating was prepared according to Example 1.
11) Multi-step grafting method: NE was dissolved in a Tris solution (pH=8.5, 1.21 mg/mL) under alkaline conditions, and a dissolved NE (3 mg/mL) was subjected to reaction on a titanium sheet for 24 h at ambient temperature (25° C.) in aerobic environment, and then a resulting system was subjected to ultrasonic cleaning and blow-drying. Further, a dissolved ZnCl₂ (1 mg/mL) in a Tris solution was subjected to reaction on the titanium sheet for 24 h under the same pH, temperature, and oxygen conditions, and then a resulting system was subjected to ultrasonic cleaning and blow-drying. Finally, a dissolved bacitracin (0.25 mg/mL) in a Tris solution was subjected to reaction on the titanium sheet for 24 h under the same pH, temperature, and oxygen conditions, and then a resulting system was subjected to ultrasonic cleaning and blow-drying to obtain an NE+Zn+bacitracin antibacterial coating.

### 2. Evaluation of In Vitro Antibacterial Properties

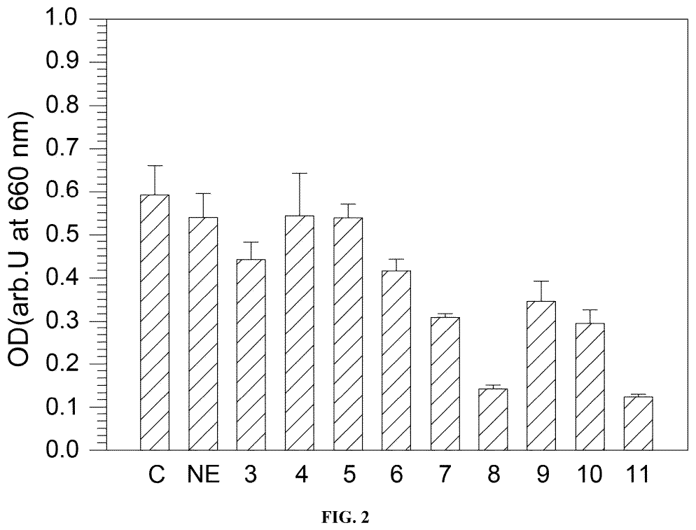

The anti-Staphylococcus aureus performance of the prepared coatings was evaluated by turbidity method. C represents the blank control, NE represents the NE coating, 3 represents the NE@Zn coating, 4 represents the NE@Zn+bacitracin antibacterial coating, 5 represents the NE+bacitracin coating, 6 represents the NE@bacitracin coating, 7 represents the NE@bacitracin+Zn antibacterial coating, 8 represents the NE@bacitracin@Zn antibacterial coating, 9 represents the NE+bacitracin coating, 10 represents the NE+bacitracin+Zn antibacterial coating, and 11 represents the NE+Zn+bacitracin antibacterial coating. As shown in FIG. 2, coatings 8 and 11 exhibit a significant antibacterial property against Staphylococcus aureus. Form the results, it can be concluded that: NE@Zn+bacitracin coating exhibits no antibacterial property, while NE+Zn+bacitracin coating exhibits an antibacterial property, which may be due to the fact that the NE@Zn blend could wrap Zn, making it difficult to achieve antibacterial effect, and the NE+Zn+bacitracin coating has a large amount of Zn on the surface of the coating, and thus exhibits a high antibacterial property; neither NE@bacitracin+Zn coating nor NE+bacitracin+Zn coating has an antibacterial property, which may be due to the fact that without the addition of Zn, it is difficult to load a large amount of bacitracin on the NE surface. The one-step blending method does not cause the problem of bacitracin and Zn being wrapped and unable to be released, and thus the coatings prepared by the same show a desirable antibacterial property.

### Example 3

Since the one-step blending method has advantages of simple operation and shortened preparation process, in the present disclosure, the antibacterial property based on the one-step blending method was further studied.

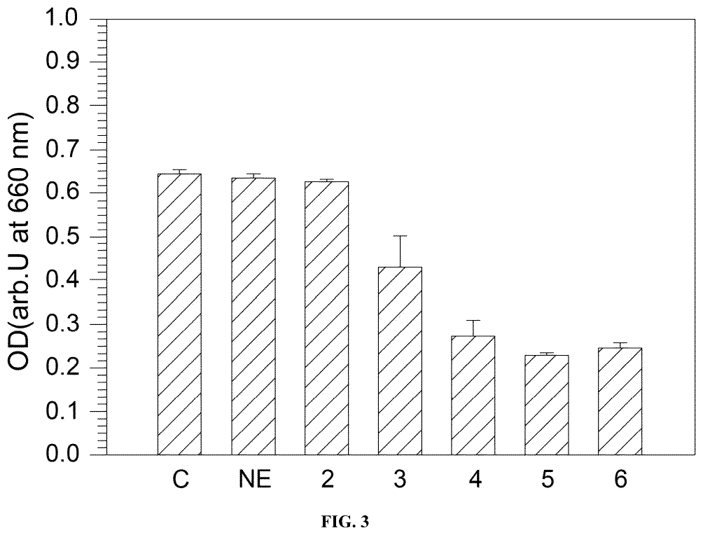

The NE@bacitracin@Zn antibacterial coatings were prepared by using bacitracin at a concentration of 0.125 mg/mL, 0.25 mg/mL, 0.5 mg/mL, and 1 mg/mL separately, according to the process described in Example 1. The anti-Staphylococcus aureus performance of these prepared coatings was evaluated using turbidity method. As shown in FIG. 3, the antibacterial effect became stable after the bacitracin concentration is increased to 0.25 mg/mL.

Furthermore, the biocompatibility of the NE@bacitracin@Zn antibacterial coatings prepared by the one-step blending method was evaluated. In previous studies, the NE coating exhibits a high biocompatibility. The biocompatibility of the coatings with different concentrations of bacitracin and Zn was investigated.

The NE@bacitracin@Zn antibacterial coatings were prepared by using bacitracin at a concentration of 0.125 mg/mL, 025 mg/mL, and 0.5 mg/mL separately, according to the process described in Example 1.

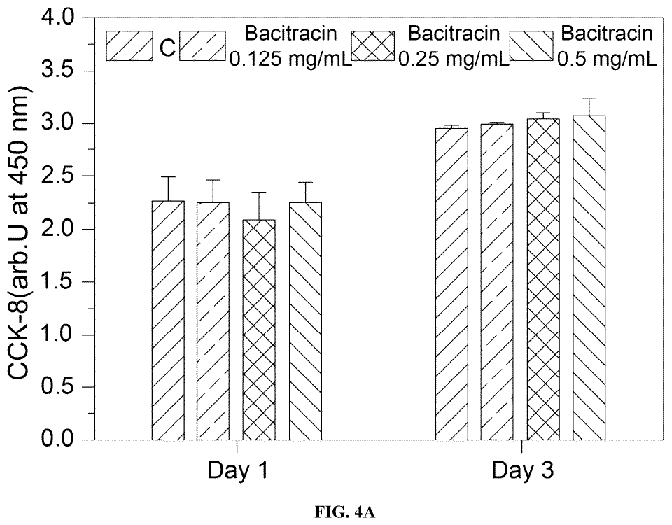

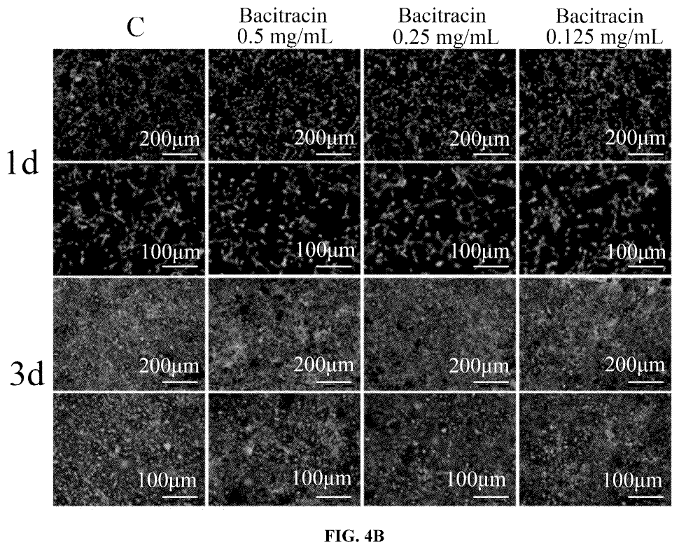

BMSC/MC3T3-E1 was used as the experimental material for cell counting kit-8 (cck-8) detection, the number of cell growth was quantitatively detected, and the OD values at 1 day and 3 days were measured, respectively. Furthermore, the cell morphology was observed by fluorescent staining, and the cells were cultured for 1 day and 3 days separately and fluorescence photos were then taken. As shown in FIG. 4A, the NE@bacitracin@Zn antibacterial coatings prepared by using 0.125 mg/mL, 0.25 mg/mL, and 0.5 mg/mL bacitracin all show a desirable biocompatibility; the results of fluorescence staining and cck-8 detection are consistent, as shown in FIG. 4B. For economic reasons, the concentration of bacitracin is selected to be 0.25 mg/mL.

Further, the influence of Zn concentration on biocompatibility was studied.

The NE@bacitracin@Zn antibacterial coatings were prepared by using ZnCl₂ at a concentration of 0.25 mg/mL, 0.5 mg/mL, and 1 mg/mL separately, according to the process described in Example 1.

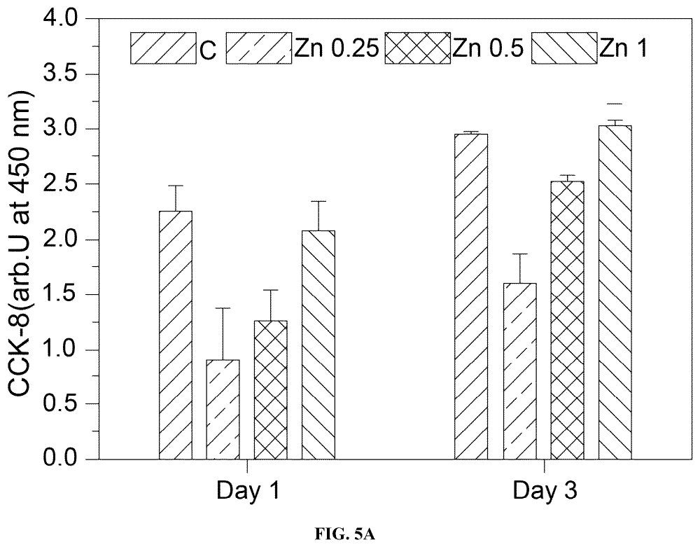

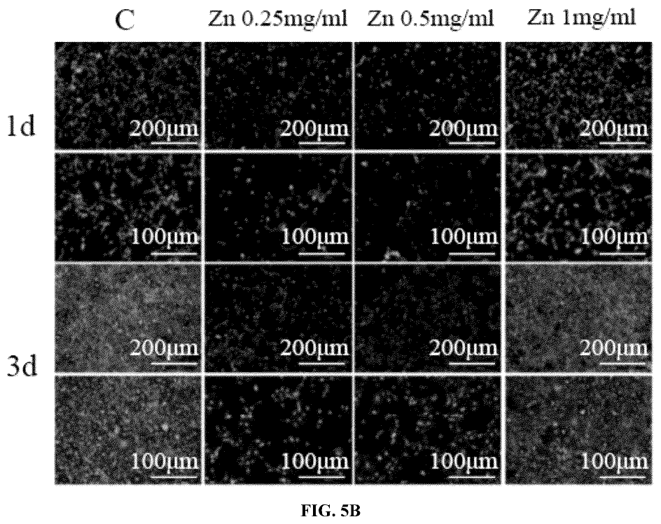

BMSC/MC3T3-E1 was used as the experimental material for cell counting kit-8 (cck-8) detection, the number of cell growth was quantitatively detected, and the OD values at 1 day and 3 days were measured, respectively. Furthermore, the cell morphology was observed by fluorescent staining, and the cells were cultured for 1 days and 3 days separately and fluorescence photos were then taken. As shown in FIG. 5A, the NE@bacitracin@Zn antibacterial coatings prepared by using 0.25 mg/mL and 0.5 mg/mL ZnCl₂ show a toxicity, while the NE@bacitracin@Zn antibacterial coating prepared with 1 mg/ml ZnCl₂ shows a desirable biocompatibility. The results of fluorescence staining method and cck-8 detection are consistent, as shown in FIG. 5B. In the present disclosure, the preferred concentration of ZnCl₂ used in the antibacterial coating is 1 mg/mL.

The foregoing is detailed description of the preferred specific embodiments of the present disclosure. It should be understood that a person of ordinary skill in the art can make various modifications and variations according to the concept of the present disclosure without creative efforts. Therefore, all technical solutions that a person skilled in the art can obtain based on the prior art through logical analysis, reasoning, or finite experiments according to the concept of the present disclosure shall fall within the scope defined by the appended claims.

1. An antibacterial coating, which is formed by polymerization of catecholamine, bacitracin, and a metal ion.

2. A method for preparing an antibacterial coating, comprising dissolving catecholamine, bacitracin, and a metal ion in a tris(hydroxymethyl)aminomethane solution (Tris solution) to obtain a dissolved mixture, subjecting the dissolved mixture to reaction on a titanium sheet for 24 h at ambient temperature under an aerobic environment, then subjecting a surface of the titanium sheet to ultrasonic cleaning to remove a precipitate thereon, and conducting blow-drying to obtain the antibacterial coating.

3. The method according to claim 2, wherein the Tris solution has a pH value of 7.5 to 9.5.

4. The method according to claim 2, wherein the aerobic environment is achieved by exposure to air.

5. The method according to claim 2, wherein the catecholamine is norepinephrine (NE), and the metal ion is provided by ZnCl₂.

6. The method according to claim 5, wherein in the dissolved mixture, the NE has a concentration of 1 mg/mL to 5 mg/mL, the bacitracin has a concentration of 0.12 mg/mL to 1 mg/mL, and the ZnCl₂ has a concentration of 0.5 mg/mL to 2 mg/mL.

7. The method according to claim 6, wherein in the dissolved mixture, the NE has a concentration of 3 mg/mL, the bacitracin has a concentration of 0.25 mg/mL, and the ZnCl₂ has a concentration of 1 mg/mL.
